(19) United States
(12) Patent Application Publication
(10) Pub. No.: US 2016/0184279 A1
(43) Pub. Date: Jun. 30, 2016

(54) METHODS OF TREATING PAIN AND/OR ITCH WITH SMALL MOLECULE INHIBITORS TARGETING AN mTOR PATHWAY

(71) Applicant: Transderm, Inc., Santa Cruz, CA (US)

(72) Inventors: Roger L. Kaspar, Santa Cruz, CA (US); Tycho Speaker, Santa Cruz, CA (US)

(73) Assignee: Transderm, Inc., Santa Cruz, CA (US)

(21) Appl. No.: 14/965,839

(22) Filed: Dec. 10, 2015

Related U.S. Application Data
(60) Provisional application No. 62/090,286, filed on Dec. 10, 2014.

(51) Int. Cl. A61K 31/436; A61K 9/00

(52) U.S. Cl. CPC A61K 31/436; A61K 9/0014

(57) ABSTRACT

Methods of treating pain and/or itch in a targeted region of a subject and compositions and dosage forms therefor are described herein. Such methods can include topically administering a therapeutically effective amount of an mTOR pathway inhibitor to the subject.

### PRIORITY DATA

This application claims the benefit of U.S. Provisional Application Ser. No. 62/090,286 filed on Dec. 10, 2014, which is incorporated herein by reference.

### BACKGROUND

Rapamycin (Sirolimus) is a macrocyclic lactone that has been known as a pharmaceutically active agent for use in treating a variety of conditions. Various related compounds, such as temsirolimus, everolimus, BEZ-235, GSK2126458, torin-1, torin-2, WYE-354, and metformin show similar activity, suppressing the mammalian target of rapamycin (mTOR) biochemical pathway. RAPAMUNE®, an FDA approved dosage form, is an oral dosage form of rapamycin.

### DETAILED DESCRIPTION OF EXAMPLE EMBODIMENTS

Although the following detailed description contains many specifics for the purpose of illustration, a person of ordinary skill in the art will appreciate that many variations and alterations to the following details can be made and are considered to be included herein. Such alterations or variations may become apparent after a review of the present application. Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

As used in this specification and the appended claims, the singular forms “a,” “an” and “the” include plural referents unless the context clearly dictates otherwise. Thus, for example, reference to “a cell” includes a plurality of such cells.

Occurrences of the phrase “in one embodiment,” or “in one aspect,” or “in one example,” herein do not necessarily all refer to the same embodiment, aspect, or example.

As used herein, “subject” refers to a mammal that can benefit from treatment with an mTOR inhibitor. A benefit can be obtained if the subject has a disease or condition, or is at risk of developing a disease or condition for which an mTOR inhibitor is a therapeutically effective treatment or preventative measure. In some aspects, such subject may be a human.

As used herein, the terms “treat,” “treatment,” or “treating” when used in conjunction with the administration of an mTOR inhibitor, such as rapamycin, including compositions and dosage forms thereof, refers to administration to subjects who are either asymptomatic or symptomatic. In other words, “treat,” “treatment,” or “treating” can be to reduce, ameliorate or eliminate symptoms associated with a condition present in a subject, or can be prophylactic, (i.e. to prevent or reduce the occurrence of the symptoms in a subject). Such prophylactic treatment can also be referred to as prevention of the condition. Treatment outcomes can be expected or unexpected. In one specific aspect, a treatment outcome can be a delay in occurrence or onset of a disease or conditions or the signs or symptoms thereof. In another aspect, a treatment can be reducing, ameliorating, eliminating, or otherwise providing a subject with relief from (i.e. relieving) the condition with which they are afflicted, or providing relief from signs or symptoms of the condition.

The term “mTOR inhibitor” refers to agents or compounds which are effective in inhibiting mTOR or an inhibitor of the mTOR signaling pathway. mTOR is a serine/threonine kinase that regulates translation and cell division. Examples of mTOR inhibitors include but are not limited to rapamycin (sirolimus) and its analogues and derivatives (Rapalogs), everolimus, ridaforolimus, and temsirolimus, as well as Torinl, Torin2, PI-103, NVP-BEZ235, NVP-BTG226, GSK1059615, GSK2126458, GDC-0980, GNE-477, XL765, PKI-402, PF-05212384, PF-04691502, WJD008, AZD-8055, AZD-2014, OSI-027, INK-128, Ku0063794, OXA-01, Palomid 529, TorkiCC2323, WAY600, WYE132, WYE353, WYE687, the like, and combinations thereof.

As used herein, “rapamycin” or “sirolimus” are used interchangeably and refer to the macrocyclic lactone produced by the organism Streptomyces hydroscopicus isolated from soil samples of Easter Island (Rapa Nui) and having the structure of:

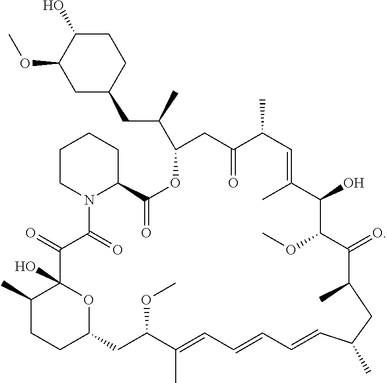 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

As used herein, “rapamycin and/or other mTOR inhibitors”, refer to the primary mTOR inhibitor, rapamycin itself, and also to related compounds such as and various related compounds, such as temsirolimus, everolimus, torin-1, torin-2, WYE-354, and metformin, which may have similar activity.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients. Compositions can take nearly any physical state, including solid, liquid (i.e. solution), or gas. Furthermore, the term “dosage form” can include one or more formulation(s) or composition(s) provided in a format for administration to a subject. In one example, a composition can be a preparation that releases or otherwise administers an mTOR inhibitor.

As used herein, “effective amount,” “therapeutically effective amount,” “pain-treating amount,” or “itch-treating amount” of an mTOR inhibitor, such as rapamycin, refers to a sufficient amount or concentration of an mTOR inhibitor in a formulation or composition to provide an intended effect and/or achieve an intended result when administered to a subject. For example, a therapeutically effective amount of rapamycin may be an amount which is sufficient to treat a particular target indication, e.g. topical pain and/or itch, local pain and/or itch, systemic pain and/or itch or other condition for which an mTOR inhibitor can be used. It is understood that various biological factors may affect the ability of a particular agent to perform its intended task. Therefore, an “effective amount” or a “therapeutically effective amount” or “itch-treating amount” or “pain-treating amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986).

As used herein, “skin,” “skin surface,” “derma,” “epidermis,” and similar terms are used interchangeably, and refer to not only the outer skin of a subject comprising the epidermis, but also to underlying layers and to mucosal surfaces.

As used herein, a “dosing regimen” or “regimen” such as “treatment dosing regimen,” or a “prophylactic dosing regimen,” refers to how, when, how much, and for how long a dose of a composition can or should be administered to a subject in order to achieve an intended treatment or effect.

As used herein, the terms “release” and “release rate” are used interchangeably to refer to the discharge or liberation, or rate thereof, of a substance, including without limitation a therapeutic agent, such as an mTOR inhibitor, from the dosage form or composition containing the substance. In one example, a therapeutic agent may be released in vitro. In another aspect, a therapeutic agent may be released in vivo.

As used herein, “immediate release” or “instant release” can be used interchangeably and refer to immediate or near immediate (i.e. uninhibited or unrestricted) release of an agent or substance, including a therapeutic agent, such as an mTOR inhibitor, from a composition or formulation.

As used herein, the term “controlled release” refers to non-immediate release of an agent or substance, including a therapeutic agent, such as an mTOR inhibitor, from a composition or formulation. Examples of specific types of controlled release include without limitation, extended or sustained release and delayed release. Any number of control mechanisms or components can be used to create a controlled release effect, including formulation ingredients or constituents, formulation properties or states, such as pH, an environment in which the formulation is placed, or a combination of formulation ingredients and an environment in which the formulation is placed. In one example, extended release can include release of a therapeutic agent at a level that is sufficient to provide a therapeutic effect or treatment for a non-immediate specified or intended duration of time.

As used herein the term “topical formulation” refers to a formulation that may be applied to skin or a mucosa. Topical formulations may, for example, be used to treat a subject by delivering an active agent or drug, such as an mTOR inhibitor. Topical formulations can be used for both topical and transdermal administration of substances. Examples of topical formulations include but are not limited to ointments, creams, lotions, gels, and pastes.

As used herein, “topical administration” is used in its conventional sense to mean delivery of a substance, such as a therapeutically active agent, to the skin or a localized region of a subject's body. Topical administration of a drug, such as an mTOR inhibitor may often be advantageously applied in, for example, the treatment of pain and/or itch in a subject's skin. While topical administration can be for the purpose of treating a local area or region of tissue, such as skin, topical administration can also be for the purpose of providing transdermal administration.

As used herein, “transdermal administration” refers to administration through the skin. Transdermal administration is often applied where systemic delivery of an active is desired, although it may also be useful for delivering an active to tissues underlying the skin with minimal systemic absorption.

As used herein, “penetration enhancer” refers to an agent that improves the transport of molecules such as an active agent (e.g., a drug) into or through the skin. Various conditions may occur at different sites in the body either in the skin or below creating a need to target delivery of compounds. Thus, a “penetration enhancer” may be used to assist in the delivery of an active agent directly to the skin or underlying tissue or indirectly to the site of the disease or a symptom thereof through systemic distribution. A penetration enhancer may be a pure substance or may comprise a mixture of different chemical entities.

As used herein, “carrier,” and “pharmaceutically acceptable carrier” may be used interchangeably, and refer to any liquid, gel, salve, solvent, liquid, diluent, fluid ointment base, liposome, micelle, giant micelle, and the like, which is suitable for use in contact with a subject or it's tissue without causing adverse physiological responses, and which does not interact with the other components of the composition in a deleterious manner. A number of carrier ingredients are known for use in making topical formulations, such as gelatin, polymers, fats and oils, lecithin, collagens, alcohols, water, etc.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” in this specification it is understood that direct support should be afforded also to “consisting essentially of” language as well as “consisting of” language as if stated explicitly and vice versa.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, compounds, formulations, or other items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. Numerical values recited herein as weight percent (wt %) are presumed to be with respect to a formulation or composition in which an ingredient associated with the numerical value is included, unless the context specifically dictates otherwise.

This same principle applies to ranges reciting only one numerical value as a minimum or a maximum. Furthermore, such an interpretation should apply regardless of the breadth of the range or the characteristics being described.

Reference in this application may be made to compositions, systems, or methods that provide “improved” performance. It is to be understood that unless otherwise stated, such “improvement” is a measure of a benefit obtained based on a comparison to compositions, systems or methods in the prior art. Furthermore, it is to be understood that the degree of improved performance may vary between disclosed embodiments and that no equality or consistency in the amount, degree, or realization of improvement is to be assumed as universally applicable.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

Unless otherwise defined, all technical and scientific terms used herein have the same meaning as commonly understood to one of ordinary skill in the art to which this invention belongs. Although any methods, compositions, devices and materials similar or equivalent to those described herein can be used in the practice or testing of the invention, representative methods, devices, and materials are described below.

### Example Embodiments

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

mTOR inhibitors are a potent class of pharmaceutically active agents that can be useful in treating a variety of conditions or symptoms. Two examples of such symptoms are pain and itch. mTOR inhibitors can be administered in a variety of ways, including, but not limited to, oral, topical, intravenous, intrathecal, and transdermal administration. Therefore, mTOR inhibitors can be used to treat a wide range of pain and/or itch symptoms both systemically and in targeted regions or areas of a subject's body.

For example, a subject may experience topical pain and/or itch due to a skin disease, disorder, condition, allergy (e.g. an allergic reaction), or injury. Accordingly, an mTOR inhibitor can be administered as a first line of treatment to alleviate the pain and/or itch. When pain and/or itch symptoms are manifested in the skin, it may be desirable to apply treatment directly to the situs afflicted with these symptoms. In instances where the disease or condition is one afflicting the skin, it is possible that the pain and/or itch symptoms can share a localized region with the condition. In such cases, the topical administration can also be made to the situs of the disease or condition in order to treat the pain and/or itch symptoms and may in some instances also treat the underlying condition. When the disease or condition causing the pain and/or itch symptoms in the skin, is not a skin condition, the administration of the topical formulation will be at the targeted region containing the situs of the pain and/or itch, but not at the situs of the disease or condition.

In one aspect, a topical formulation containing a therapeutically effective amount of an mTOR inhibitor can be used to treat the symptoms of pain and/or itch at a targeted localized region or area of subject's skin by direct application thereto. Numerous skin conditions produce the symptoms of pain and/or itch. While some occur in a single localized area, others may manifest these symptoms in a number of localized areas. Examples of skin conditions can include without limitation, keratoses such as tuberous sclerosis, seborrheic keratosis, keratosis pilaris, epidermolysis bullosa, multiple minute digitate hyperkeratosis, hyperkeratosis lenticularis perstans, stasis dermatitis, focal acral hyperkeratosis, follicular hyperkeratosis, lichenoid keratoses including lichen planus and lichen sclerosus, Conradi-Eltinermann, epidermolytic ichthyosis, erythrokeratoderma variabilis, ichthyosis hystrix, KID syndrome, Netherton syndrome, Olmsted syndrome, Refsum disease, Sjogren-Larsson Syndrome, and actinic keratosis, pachyonychia congenita, hyperhidrosis, warts, calluses, and dermatitis such as contact dermatitis, drug-induced dermatitis, allergic dermatitis, nummular dermatitis, perioral dermatitis, neurodermatitis, seborrheic dermatitis, and atopic dermatitis, psoriasis, acne, carbunculosis, cellulitis, furunculosis, granuloma, acanthosis nigricans, athlete's foot, bacterial vaginosis, balanitis, cancer and tumor phemomena, including dermatofibrosarcoma protruberans, basal cell carcinoma, squamous cell carcinoma, melanoma, merkel cell carcinoma, and keloid, cysts and tumors including lymphangioma, venous malformation, epidermal nevi, bromhidrosis, dermatophytosis such as candidiasis, onychomycosis, tinea including tinea alba, tinea pedis, tinea unguium, tinea manuum, tinea cruris, tinea corporis, tinea capitis, tinea faciei, tinea barbae, tinea imbricata, tinea nigra, tinea versicolor, and tinea incognito, eczema such as dyshydrotic eczema, decubitous ulcer, drug reactions, ecthyma, erysipalus, erythema multiforme, impetigo, insect bites, genital warts, hemangioma, herpes virus such as herpes simplex, herpes stomatitis, and herpes zoster, hives, hyperhidrosis, filariasis, lentigines, lupus, miliaria, milker's nodules, molluscum contagiosum, myiasis such as scabies, cutaneous larva migrans, furuncular myiasis, migratory myiasis, and pediculosis, nevus araneus, panniculitis, paronychia, pemphigoid, pityriasis, pruritis vulvae, rosacea, trichomoniasis, vaginal yeast infection, vitiligo, xeroderma.

In addition, numerous skin injuries produce symptoms of pain and/or itch, which can be treated using the formulations of the present invention. Non-limiting examples include, burns, such as sunburns, bites, such as insect bites, chemical reactions, localized allergic reactions, and abrasions, whether purposefully or non-purposefully induced. One example of a purposefully induced injury would be a chemical peel. Another would be a closed, but not yet healed surgical incision. Other medical procedures which remove one or more layers of the skin are also considered to be purposeful injuries. While many injuries will be localized, others may be more widely spread such as sunburn. An example of a chemical reaction could be contact with an irritant, such as an industrial product, or a plant irritant, such as poison ivy, poison oak, or sumac.

In yet other embodiments, pain and/or itch symptoms may manifest in the skin either systemically, or at localized regions due to a condition or disease that is away from the area of pain and/or itch symptom manifestation. In such cases, the targeted regions will be the locations where the pain and/or itch symptoms occur and the formulations of the present invention can be administered thereto. In such cases, administration of the present formulations will typically treat only the pain and/or itch symptoms, and not the underlying condition since the situs of the disease or condition is outside of the targeted region. One exception is when the underlying condition is one that also responds to treatment with an mTOR inhibitor, and the amount of mTOR inhibitor in the formulation is therapeutically effective in treating both the pain and itch symptoms manifest on the skin, as well as transdermally reach and treat the underlying condition. Examples of such conditions can include without limitation, keratoses such as tuberous sclerosis, seborrheic keratosis, keratosis pilaris, epidermolysis bullosa, multiple minute digitate hyperkeratosis, hyperkeratosis lenticularis perstans, stasis dermatitis, focal acral hyperkeratosis, follicular hyperkeratosis, lichenoid keratoses including lichen planus and lichen sclerosus, Conradi-Hunermann, epidermolytic ichthyosis, erythrokeratoderma variabilis, ichthyosis hystrix, KID syndrome, Netherton syndrome, Olmsted syndrome, Refsum disease, Sjogren-Larsson Syndrome, and actinic keratosis, pachyonychia congenita, hyperhidrosis, warts, calluses, and dermatitis such as contact dermatitis, drug-induced dermatitis, allergic dermatitis, nummular dermatitis, perioral dermatitis, neurodermatitis, seborrheic dermatitis, and atopic dermatitis, psoriasis, acne, carbunculosis, cellulitis, furunculosis, granuloma, acanthosis nigricans, athlete's foot, bacterial vaginosis, balanitis, cancer and tumor phemomena, including dermatofibrosarcoma protruberans, basal cell carcinoma, squamous cell carcinoma, melanoma, merkel cell carcinoma, and keloid, cysts and tumors including lymphangioma, venous malformation, epidermal nevi, bromhidrosis, dermatophytosis such as candidiasis, onychomycosis, tinea including tinea alba, tinea pedis, tinea unguium, tinea manuum, tinea cruris, tinea corporis, tinea capitis, tinea faciei, tinea barbae, tinea imbricata, tinea nigra, tinea versicolor, and tinea incognito, eczema such as dyshydrotic eczema, decubitous ulcer, drug reactions, ecthyma, erysipalus, erythema multiforme, impetigo, insect bites, genital warts, hemangioma, herpes virus such as herpes simplex, herpes stomatitis, and herpes zoster, hives, hyperhidrosis, filariasis, lentigines, lupus, miliaria, milker's nodules, molluscum contagiosum, myiasis such as scabies, cutaneous larva migrans, furuncular myiasis, migratory myiasis, and pediculosis, nevus araneus, panniculitis, paronychia, pemphigoid, pityriasis, pruritis vulvae, rosacea, trichomoniasis, vaginal yeast infection, vitiligo, xeroderma.

In other instances, the pain may not be manifest at the skin, but rather at an internal location of the body, such as an organ, muscle, bone, or other connective tissue. When this is the case, the compositions of the present invention can be formulated to provide transdermal administration of a therapeutically effective amount of an mTOR inhibitor in order to allow the drug to reach the condition situs and provide pain relief.

Various factors can affect an appropriate amount of an mTOR inhibitor to ameliorate the pain and/or itch symptoms in a subject. Such factors can include the specific mTOR inhibitor or inhibitors being used, type of pain-producing or itch-producing condition experienced by the subject, the age and weight of the subject, as well as various other physical and genetic factors, other medications being used to treat the patient, and many other factors known by those skilled in the relevant arts. As a result, there are a range of therapeutically effective amounts that can be used for treating the pain and/or itch of a subject, which can depend on the above listed factors and others. In one aspect, a therapeutically effective amount can be a pain-treating amount. In another aspect, a therapeutically effective amount can be an itch-treating amount. In another aspect, a therapeutically effective amount can be both a pain-treating amount and an itch-treating amount.

In some examples, a therapeutically effective amount can include an amount from about 0.1 mg to about 10000 mg per day. In another aspect, a therapeutically effective amount can include an amount from about 1 mg to about 500 mg per day. In another aspect, a therapeutically effective amount can include an amount from about 2 mg to about 100 mg per day. In yet a further aspect, a therapeutically effective amount can include an amount from about 2 mg to about 20 mg per day.

In some examples, a dose of an mTOR inhibitor from about 1 mg to about 2000 mg can be administered topically to a subject from 1 to 6 times per day to treat the symptoms of pain and/or itch. In some examples, a dose of an mTOR inhibitor from about 10 mg to about 1000 mg can be administered topically to a subject from 1 to 6 times per day to treat the symptoms of pain and/or itch. In some examples, a dose of from about 10 mg to about 500 mg can be administered topically to a subject from 1 to 6 times per day to treat the symptoms of pain and/or itch. In one specific example, a topical delivery vehicle can include 20 wt % of an mTOR inhibitor. In some cases, the delivery vehicle can be applied topically to the skin of a subject in an amount of about 30 g up to 6 times per day. Accordingly, about 1.5 g of an mTOR inhibitor can be administered per individual dose for a total daily dose of about 9 g. In another specific example, a topical delivery vehicle can include 1 wt % of an mTOR inhibitor. In some cases, the delivery vehicle can be applied topically to the skin of a subject in an amount of about 10 g up to 6 times per day. Accordingly, about 100 mg of an mTOR inhibitor can be administered per individual dose for a total daily dose of about 600 mg. Many other dosage amounts can be used as well. For example, the delivery vehicle can be applied in amounts from about 0.05 g to about 30 g, or from about 0.1 g to about 20 g, or from about 0.1 g to about 10 g, or from 0.1 g to about 1 g, or combinations thereof.

In one embodiment, the amount of mTOR inhibitor that is therapeutically effective may be sufficient to provide a reduction of pain severity; delay of onset of severe pain following stimuli; accelerate recovery from pain following stimuli; reduce burning sensation; reduce reduction of itching sensation, especially deep itch. When applied to a target region which also includes a condition or disease that causes the pain and/or itch symptoms, the amount of mTOR inhibitor can also be sufficient to provide thinning of hyperkeratosis; reduction of toughness/softening of hyperkeratosis, reduction of growth rate of hyperkeratosis, reduction of overheating sensation, reduction of superficial blood vessels, and reduction of blistering/cracking, among other things.

As previously mentioned, the therapeutically effective amount can be affected by which mTOR inhibitor or inhibitors are being used to treat the pain and/or itch. A variety of mTOR inhibitors can be used, either alone or in combinations. Such mTOR inhibitors can include at least one of rapamycin, temsirolimus, everolimus, AP-23573, AP-23481, analogues thereof, derivatives thereof, and combinations thereof. In one aspect, the pain and/or itch can be treated using rapamycin or an analogue thereof.

A variety of topical formulations can be used in order to administer a dose of an mTOR inhibitor to the skin. Exemplary formulations can include can include a patch, a gel, a cream, an ointment, a lotion or other topical or transdermal delivery system.

Formulations for topical or transdermal delivery systems which effectively administer mTOR inhibitors to treat pain can be challenging to make. One such challenge can be caused by the structures of the various mTOR inhibitors and the associated solubility and/or stability challenges that exist for many of these compounds. For example, rapamycin has intermediate polarity and is therefore poorly solubilized in aqueous compositions, ethanolic compositions, triglycerides, alkanes, and silicone systems. Benzyl alcohol has been identified as a solvent that can effectively solubilize rapamycin. In particular, benzyl alcohol can support 20 wt % solutions and higher of rapamycin. However, benzyl alcohol does not conveniently form a stable emulsion in formulations using typical pharmaceutical surfactants where benzyl alcohol is at a concentration above 10 wt %. For example, formulations utilizing Span 80 and/or Tween 20 as surfactants were only able to achieve benzyl alcohol loading of 20 wt % or less. Further, emulsions made with Span 80 and/or Tween 20 demonstrated poor stability during overnight storage at ambient temperature, producing 5% separation with discrete coalesced droplets being visible.

With the above in mind, a stable and effective formulation or composition for administering mTOR inhibitors to treat pain and/or itch can include a therapeutically effective amount of an mTOR inhibitor, a polymer having surfactant properties, a polymer having thickening properties, a solvent for solubilizing the mTOR inhibitor, a glycol, a C₁₀-C₂₀ fatty acid, a base, and water.

In one specific example, a formulation for topical application of an mTOR inhibitor can include rapamycin, a polymer having surfactant properties, a polymer having thickening properties, benzyl alcohol, a glycol, a C₁₀-C₂₀ fatty acid, a base, and water. In a further embodiment, the transdermal delivery system can include from about 55 wt % to about 98 wt % water, about 0.05 wt % to about 5.0 wt % rapamycin, about 0.05 wt % to about 1.0 wt % of a polymer having surfactant properties, about 0.1 wt % to about 2 wt % of a polymer having thickening properties, about 0.5 wt % to about 5.0 wt % of a glycol, about 0.1 wt % to about 10 wt % of benzyl alcohol, about 0.2 wt % to about 2.5 wt % of a C₁₀-C₂₀ fatty acid, and about 0.01 wt % to about 0.5 wt % of a base.

As previously mentioned, the therapeutically effective amount of an mTOR inhibitor present in a topical or transdermal delivery system can vary depending on the particular mTOR inhibitor being used. In one embodiment, the delivery system can include from 0.01 wt % to about 20 wt % of an mTOR inhibitor. In another embodiment, the delivery system can include about 0.05 wt % to about 10 wt % of the formulation. In another embodiment, the mTOR inhibitor can comprise about 0.05 wt % to about 8 wt %. In another embodiment, the mTOR inhibitor can comprise about 0.05 to about 5 wt %. In still a further embodiment, the mTOR inhibitor can comprise about 0.1 to about 3 wt %. In one embodiment, the mTOR inhibitor can comprise 0.1 wt % to about 2 wt % of the formulation. In still a further embodiment, the mTOR inhibitor can comprise about 1 wt % of the formulation. In one example, the mTOR inhibitor can be rapamycin.

Polymers having surfactant properties (surfactant polymers) can include a wide array of surfactant or emulsifying polymers that are known in the art. Non-limiting examples of polymers having surfactant or emulsifying properties include, but are not limited to acrylate C₁₀-C₃₀ alkyl acrylate crosspolymers, hydrophobically modified polyacrylic acid commercially available under the tradename Pemulen™ TR-I and TR-2 by Lubrizol Corp., water-soluble or water-swellable copolymers based on acrylamidoalkyl sulfonic acid and cyclic N-vinylcarboxamides commercially available under the tradename Aristoflex® AVC by Clariant Corporation; water-soluble or water-swellable copolymers based on acrylamidoalkyl sulfonic acid and hydrophobically modified methacrylic acid commercially available under the tradename Aristoflex® HMB by Clariant Corporation and a homopolymer of acrylamidoalkyl sulfonic acid commercially available under the tradename Granthix APP by Grant Industries, Inc. Another class of notable polymeric emulsifiers includes hydrophobically-modified, crosslinked, anionic acrylic copolymers, including random polymers, but may also exist in other forms such as block, star, graft, and the like. In one embodiment, the hydrophobically modified, crosslinked, anionic acrylic copolymer may be synthesized from at least one acidic monomer and at least one hydrophobic ethylenically unsaturated monomer. Examples of suitable acidic monomers include those ethylenically unsaturated acid monomers that may be neutralized by a base. Examples of suitable hydrophobic ethylenically unsaturated monomers include those that contain a hydrophobic chain having a carbon chain length of at least about 3 carbon atoms.

Other materials that may be suitable polymeric surfactants can include ethylene oxide/propylene oxide block copolymers, sold under the trade name PLURONIC®, available from BASF Corporation of Parsippany, N.J., modified cellulose polymers such as those modified cellulose polymers described by the trade name KLUCEL®, available from Hercules Corporation of Wilmington, Del. Particularly notable embodiments of the invention are compositions that include hydrophobically modified polyacrylic acid, acrylamidoalkyl sulfonic acid, cyclic N-vinylcarboxamides, acrylamidoalkyl sulfonic acid, hydrophobically modified methacrylic acid, a homopolymer of acrylamidoalkyl sulfonic acid, or combinations thereof as polymeric emulsifiers; and monomeric anionic surfactants, monomeric amphoteric surfactants, or combinations thereof as foaming agents. More particularly notable embodiments of the invention are compositions that include hydrophobically modified polyacrylic acid; water-soluble or water-swellable copolymers based on acrylamidoalkyl sulfonic acid, cyclic N-vinylcarboxamides; water-soluble or water-swellable copolymers based on acrylamidoalkyl sulfonic acid, hydrophobically modified methacrylic acid; a homopolymer of acrylamidoalkyl sulfonic acid, or combinations thereof as polymeric emulsifiers, and include a betaine as the foaming surfactant. Especially notable embodiments of the invention are compositions that include copolymers based on acrylamidoalkylsulfonic acids and cyclic N-vinylcarboxamides and/or linear N-vinylcarboxamides (e.g., Aristoflex® AVC and Aristoflex® HMB from Clariant Corporation) as polymeric emulsifiers and a betaine as foaming surfactant.

Polymers having surfactant properties can enhance the ability of a transdermal delivery system to support highly loaded emulsions of low polarity oils. For example, polymers having surfactant properties can emulsify an intermediate polarity material such as benzyl alcohol. In some cases, using common practices of predispersing surfactant polymers in a non-aqueous dispersed phase (i.e. benzyl alcohol) phase or co-dispersing the surfactant polymer with an oil phase into an aqueous phase are not capable of producing stable emulsions. Rather, the benzyl alcohol phase can wet the surfactant polymer powder and plasticize the polymer, creating “gummy” lumps that show no tendency to solubilize in a continuous aqueous phase, even with extended stirring of many hours. However, a surfactant polymer material can be fully pre-dispersed and solubilized in an aqueous phase before any benzyl alcohol is dispersed into the solution. This can substantially prevent or avoid formation of gummy lumps that otherwise form with the conventional procedure described above.

In some embodiments, the surfactant polymer can comprise about 0.01 wt % to about 3 wt %. In one embodiment, the surfactant polymer can comprise about 0.01 wt % to about 2.0 wt % of the formulations of the present invention. In one embodiment, the surfactant polymer can comprise about 0.05 wt % to about 1.0 wt % of the total formulation. In another embodiment, the surfactant polymer can comprise about 0.1 wt % to about 0.5 wt % of the total formulation.

The transdermal delivery system can also include a polymer having thickening properties (thickening polymer). In one embodiment, the polymer having thickening properties can be a hydrophobically modified cross-linked acrylate copolymer (Carbopol® Ultrez 20). Other polymers having similar properties may also be used. Non-limiting examples of polymers having thickening properties can include PEG-150 distearate, PEG-7 glyceryl cocoate, PEG-200 hydrogenated glyceryl palmitate, PEG-120 methyl glucose dioleate, carboxymethylene polymer, carboxyvinyl polymer, acrylates, C₁₀-C₃₀ alkyl acrylate crosspolymers, and combinations thereof.

Thickening polymers such as those described herein may contribute qualities to the transdermal delivery system beyond merely altering the rheology of the continuous phase. In the case that the thickening polymers exhibit solubility or plasticization of benzyl alcohol, the primary drug solvent, such polymers may influence both the solubilization of the drug to be delivered and/or the thermodynamic chemical potential of the drug and solvent system, altering the drug delivery characteristics of the formulation by a variety of related effects. Such effects can include but are not limited to: changing the evaporation rate of the drug solvent; changing the solubility limit of the drug in the solvent system, changing the solubility of the drug-solvent system in skin, changing the physical characteristics (for example freezing point) of the drug-solvent system or the continuous phase, among others. In the present composition, most preferably the polymers are selected to interact minimally with the drug-solvent system, maximizing the chemical potential of that system so as to promote rapid percutaneous delivery of the drug.

In some embodiments, the polymer having thickening properties can comprise about 0.05 wt % to about 3 wt %. In another embodiment, polymers having thickening properties can be present in amounts of 0.1 wt % to about 2 wt % of the total composition. In one embodiment, the polymer having thickening properties comprises about 0.5 wt % to about 1 wt % of the total composition. The thickening polymer can be mixed with the surfactant polymer and water as a component of an aqueous phase.

In some embodiments, the transdermal delivery system can also include a base or buffer system, which is present in the formulation to neutralize and/or activate the thickening polymer in order to facilitate the formation of a composition having the desirable rheological qualities. Any base or buffer system known in the art and suitable for use in a skin contact application can be used. In one embodiment, the base can include triethanolamine, such as solutions of 10 wt % triethanolamine (TEA), tetrasodium ethylenediaminetetraacetic acid (EDTA), alkali metal hydroxides like sodium hydroxide (NaOH), salts of weak acids such as ammonium lactate, sodium citrate, sodium ascorbate, or mixtures thereof. The base component also provides utility in that the pH of the overall composition may be adjusted to a range favorable for minimizing irritation of the skin due to pH effects. In some examples, the base can be present in an amount from 0.01 wt % to 5 wt %. In some examples, the base can be present in an amount from 0.01 wt % to 2 wt %. In some examples, the base can be present in an amount from 0.01 wt % to 0.5 wt %. In some examples, the base can be present in an amount from 0.05 wt % to 0.5 wt %. It is to be noted that where the base is present as part of a solution (e.g. 10 wt % TEA), the amount of base is to be calculated based on the proportion of base in the solution, not the total amount of solution added to the composition. By way of example, where 10 g of a 10 wt % solution of TEA in water is added to the formulation, the amount of base added to the formulation is 1 g. The other 9 g are to be attributed to the total water content of the formulation.

Additionally, in some embodiments formulations of the present invention can also include an acid or the acid component of a buffer system, and any acid known in the art and appropriate for human skin contact may be used. Examples of acids useful in the present formulation and commonly used to adjust pH of topical formulations include but are not limited to: citric acid, lactic acid, ascorbic acid, and hydrochloric acid, and combinations of these and similar acids. Whether an acid and/or base is used to adjust the pH, the pH of the formulation or delivery vehicle can be between about 4 and about 8. In another aspect, the pH of the formulation or delivery vehicle can be between about 5 and about 7.

Generally, where the pH of the formulation is between 5 and 7, the weight ratio of base to thickening polymer is in an appropriate range. In one specific example, the base can be a 10% solution of triethanolamine and the thickening polymer can be Carbopol® Ultrez 10. In some aspects, the weight ratio of TEA solution to Carbopol® polymer can be from about 1:0.6 to about 0.8:1, or from about 1:0.7 to about 0.9:1. In some cases, a weight ratio of TEA to Carbopol® outside of the ranges specified above can result in rapid and substantial breakage of the emulsion. This is because the emulsion can be highly sensitive to pH. For example, the addition of base can sometimes result in local increases of viscosity or “hot spots” of higher or lower pH that are prone to emulsion breakage. Accordingly, it can be useful to introduce the benzyl alcohol phase (rapamycin and benzyl alcohol) after the addition of the base to an initial aqueous composition including polymers, water, glycol, and fatty acid. This can also simplify the preparation of the formulation, because a freshly prepared rapamycin/benzyl alcohol solution may be added as a single component to a container in which another single component, the aqueous mixture of polymer, glycol, fatty acid and base, is pre-dispensed. The two components can then be mixed. This can be effectively accomplished by use of a “Flacktek” centrifugal mixer, similar to dental amalgam mixers, which is of particular utility in dispersing mixtures of high viscosity.

The transdermal delivery system can also include a glycol and/or glycol ether. Non-limiting examples of glycols and glycol ethers can be selected from butylene glycol, propylene glycol, diethylene glycol (Transcutol), triethylene glycol, ethylene glycol monomethyl ether, or other glycols and glycol ethers, and combinations thereof. In one aspect, the glycol and/or glycol ether can be present in the formulation in an amount from 0.1 wt % to 10 wt %. In another aspect the glycol and/or glycol ether can be present in an amount from 0.5 wt % to 5 wt %. In another aspect, the glycol and/or glycol ether can be present in an amount from 1 wt % to 4 wt %.

The transdermal delivery system can also include a C₁₀-C₂₀ fatty acid. Non-limiting examples of C₁₀-C₂₀ fatty acid can include oleic acid, arachidonic acid, linoleic acid, linolenic acid, or other fatty acids or combinations of fatty acids, and preferably unsaturated cis conformation fatty acids. Without being bound to any particular interpretation, such conformations are understood to disrupt superficial packing of the structured lipids of the stratum corneum, thereby promoting fluidization of these lipids and thus enhancing the diffusion of the drug and/or solvent into the skin, and are believed to play this role in the present formulation. In one embodiment, the C₁₀-C₂₀ fatty acid can be oleic acid. In one aspect, the C₁₀-C₂₀ fatty acid can be present in the formulation in an amount from 0.05 wt % to 10 wt %. In another aspect, the C₁₀-C₂₀ fatty acid can be present in the formulation in an amount from 0.1 wt % to 5 wt %. In another aspect, the C₁₀-C₂₀ fatty acid can be present in the formulation in an amount from 0.2 wt % to about 3 wt %. In another aspect, the C₁₀-C₂₀ fatty acid can be present in the formulation in an amount from 0.5 wt % to about 2.5 wt %.

The presence of glycol and/or fatty acid components in formulations with benzyl alcohol emulsions can sometimes result in localized breakage of the emulsion, which can be insufficiently robust at higher loadings (e.g. 25% or higher). Thus, it can be advantageous to add the glycol or fatty acid to the aqueous surfactant polymer phase (with water) in order to fully dissolve the glycol and disperse the fatty acid in the aqueous polymer phase prior to addition of the benzyl alcohol.

The presence of benzyl alcohol in the transdermal delivery system facilitates the solubility of the rapamycin. The benzyl alcohol can comprise about 0.1 wt % to about 20 wt % of the formulation. In one embodiment, the benzyl alcohol can comprises about 0.1 wt % to about 10 wt % of the formulation. In another embodiment, the benzyl alcohol can comprise about 0.5 wt % to about 8 wt % of the formulation. In another embodiment, the benzyl alcohol can comprise about 0.5 wt % to about 6 wt % of the formulation. When the rapamycin is added to the benzyl alcohol the rapamycin is solubilized in the benzyl alcohol. In one embodiment, the benzyl alcohol and the rapamycin collectively can comprise about 0.1 wt % to about 40 wt % of the formulation. In one embodiment, the formulations of the present invention can be free of components that solubilize rapamycin other than benzyl alcohol.

In some embodiments, the transdermal delivery system can also include an emollient component, for example mineral oil, dimethicone, or combinations thereof. The emollient component may provide multiple advantages, which include but are not limited to improving the cosmetic feel and appearance of the formulation during application and after drying. A wide range of emollient additives are known in the art and any of these may be included in the present compositions. Generally, inclusion of emollient materials is understood by those versed in the art to suppress evaporation rate and to reduce the chemical potential of the drug-solvent system in regard to percutaneous absorption. In one embodiment, the emollient can be present in the formulation in an amount from 0.1 wt % to 10 wt %. In another embodiment, the emollient can be present in the formulation in an amount from 0.1 wt % to 5 wt %. In another embodiment, the emollient can be present in the formulation in an amount from 0.5 wt % to 3 wt %.

Many emollients are co-soluble with the benzyl alcohol drug solvent and particular emollients may be selected to influence the delivery characteristics of the composition. For example, triglycerides are soluble in benzyl alcohol and will thereby reduce the evaporation rate and the chemical potential of the drug-solvent system in regard to percutaneous absorption. Without wishing to be bound to any particular interpretation, it is believed that because mineral oil and dimethicone are poorly miscible with benzyl alcohol, such dilution effects upon drug delivery performance can be circumvented. It is also believed that the tendency of mineral oil and silicone to form a barrier-like films on the skin surface can help to promote percutaneous absorption in a manner analogous to the effect of an occlusive dressing, well known to improve topical delivery of some drugs. Accordingly, in some embodiments, the formulation can include emollient materials that are poorly miscible with the solvent for solubilizing the mTOR inhibitor. In some cases, inclusion of such immiscible emollients can further provide improved cosmetic feel and also prevent formation of a visible powdery residue of the polymeric components, which is believed to be because of simple wetting of the residual powder by the emollient component. Formulations produced without inclusion of these immiscible emollients can leave a distinct visible white powdery deposit.

In another embodiment, the topical or transdermal delivery system can be in the form of aqueous lotions or creams. These topical or transdermal delivery systems can be such that following application to a skin surface, the skin surface is dry, or substantially dry, to the touch within about 1 minute to about 5 minutes. In one embodiment, the following application of the transdermal delivery system to a skin surface, the skin surface is dry, or substantially dry, to the touch within about 1 minute to about 2 minutes. In another embodiment, following application to a skin surface, the skin surface is dry, or substantially dry, to the touch in less than about 1 minute. In one embodiment, the formulation of the present invention can be substantially free of triglycerides, waxes, or liquid surfactants that, following application to a skin surface and being allowed to dry, are left behind on the skin surface (i.e. leave a residue). Following drying, the topical or transdermal delivery system of the present disclosure typically does not leave a residue on the skin surface. This is advantageous in that the risk of transfer of the substances, particularly the rapamycin, from the skin is significantly reduced as compared to other non-aqueous formulations (e.g. ointments). Further, by reducing superficial residue on the skin surface, the presence of materials that might solubilize rapamycin locally at the skin surface without assisting their transport onto or into the skin is reduced, which tendency might otherwise act to compromise the efficacy of the composition. For instance, if a triglyceride residue remained at the surface of the skin while the other components evaporated or absorbed into the skin, the residual triglyceride would be likely to dissolve a fraction of the rapamycin active ingredient, which would therefore be less available to be delivered by the percutaneous absorbing portions of the formulation, as topically applied triglycerides are not understood to penetrate significantly into the skin.

The compositional make-up of the topical or transdermal delivery systems disclosed herein can be such that they have a low yield value, which allows it to be readily applied to sensitive skin areas without requiring substantial pressure for rubbing or spreading. Nonetheless, the yield value of the compositions is still high enough to provide for convenient, localized, and non-messy application. This is particularly advantageous in that many conditions that can be treated with formulations of the present invention often result in tender or sensitive skin. Accordingly, the transdermal delivery systems described herein can provide for better patient compliance.

A few non-limiting examples of topically administering an mTOR inhibitor are provided.

In one example, a method of treating symptoms of pain and/or itch in a target region of a subject is described. The method can include topically administering to the subject a therapeutically effective amount of an mTOR inhibitor.

In one example, the targeted region is a local skin region.

In one example, the local skin region includes a condition producing the pain and/or itch symptom.

In one example, the condition is a member selected from the group consisting of: a keratosis, pachyonychia congenita, epidermolysis bullosa, hyperhidrosis, a wart, a callus, dermatitis, and psoriasis.

In one example, the targeted region is either a local region, a distal region, or a systemic region.

In one example, the subject is a human subject.

In one example, the mTOR inhibitor is administered locally.

In one example, the mTOR inhibitor is administered transdermally.

In one example, the therapeutically effective amount is an amount sufficient to deliver from 1 mg/day to 100 mg/day to the subject.

In one example, the therapeutically effective amount is from 0.05 wt % to 5 wt %.

In one example, the mTOR inhibitor includes at least one of rapamycin, temsirolimus, everolimus, AP-23573, AP-23481, derivatives thereof, and combinations thereof.

In one example, the mTOR inhibitor is rapamycin or an analogue thereof.

In one example, the polymer having surfactant properties is an acrylate C₁₀-C₃₀ alkyl acrylate crosspolymer.

In one example, the polymer having thickening properties is a hydrophobically modified cross-linked acrylate copolymer.

In one example, the solvent for solubilizing the mTOR inhibitor is benzyl alcohol.

In one example, the glycol is a member of the group consisting of butylene glycol, propylene glycol, diethylene glycol, triethylene glycol, ethylene glycol monomethyl ether, or other glycols and glycol ethers, and combinations thereof.

In one example, the C₁₀-C₂₀ fatty acid is a member of the group consisting of oleic acid, arachidonic acid, linoleic acid, linolenic acid, or other fatty acids or combinations thereof.

In one example, the base is a member of the group consisting of triethanolamine, tetrasodium ethylenediaminetetraacetic acid, alkali metal hydroxides such as sodium hydroxide and combinations thereof.

In one example, the delivery vehicle further comprises an emollient.

In one example, the emollient is a member of the group consisting of mineral oil, dimethicone, and combinations thereof.

In one example, a method of treating pain in targeted regions of a subject is described. The method can comprise topically administering to the subject a topical mTOR inhibitor delivery system. The topical mTOR inhibitor delivery system can comprise about 0.05 wt % to about 5 wt % rapamycin, about 0.05 wt % to about 1.0 wt % of a polymer having surfactant properties, about 0.1 wt % to about 2 wt % of a polymer having thickening properties, about 0.5 wt % to about 5 wt % of a glycol, about 0.1 wt % to about 10 wt % of benzyl alcohol, about 0.2 wt % to about 3 wt % of a C₁₀-C₂₀ fatty acid, about 55 wt % to about 98 wt % water, and about 0.01 wt % to about 0.5 wt % of a base.

In one example, the topical mTOR delivery vehicle can further include from 0.1 wt % to 5 wt % of an emollient.

In one example, the emollient is mineral oil.

### EXAMPLES

### Preparation of Topical Sirolimus 1 wt %

Approximately 4000 grams (g) water were added to a mixing vessel. Approximately 13.75 g Pemulen TR-1 polymer was added incrementally to the water while stirring until the Pemulen polymer appeared wetted out. Subsequently, about 37.75 g Carbopol Ultrez-10 polymer was added incrementally until the Carbopol polymer appeared wetted out. Next, approximately 145 g propylene glycol, 70 g oleic acid, and 47 g mineral oil were added. All ingredients were mixed to uniformity. Approximately, 35 g of 10% triethanolamine in water was then added to neutralize the polymeric components, adjust the pH to between 5 and 7, and thicken the formulation. Finally, 230 g of 20 wt % sirolimus in benzyl alcohol was added to the composition to obtain a formulation having 1 wt % sirolimus.

### Clinical Trial

A double-blinded clinical trial using topical sirolimus 1% to treat painful plantar keratoderma in genotyped pachyonychia congenita (PC) patients was performed. Subjects were assessed for eligibility and were given study drug kits. During a 2-week screening period subjects self-administered placebo on each foot (to train the subject on proper application of the study drug and generate baseline data). At the end of the screening period subjects who fulfilled all eligibility criteria were randomly assigned to receive placebo or topical sirolimus 1%. Fifteen subjects met the criteria and were enrolled in the randomized phase of the study. The subjects self-administered the study drug (i.e., either placebo or topical sirolimus 1%) according to the randomization protocol for 3 months. At the end of the 3-month randomization phase, subjects applied drug product on both feet for an additional 3 months followed by a 3-month safety evaluation period during which no product was applied. One subject dropped out for personal non-drug-related reasons.

Participants in the clinical trial reported a variety of specific benefits as part of the trial endpoints, clinician notes, and an IRB-approved post-trial survey. These benefits included: reduction of pain severity, delay of onset of severe pain following stimuli, acceleration of recovery from pain following stimuli, reduction of burning sensation, reduction of itching sensation, especially deep itch, increased activity, thinning of hyperkeratoses, reduction of toughness/softening of hyperkeratoses, reduction of growth rate of hyperkeratoses, reduction of overheating sensation, reduction of superficial blood vessels, and reduction of blistering/cracking, among others.

Patients similarly reported that these benefits were lost when the treatment was ceased. This reversion to the painful disease state can result from the cessation of therapeutic targeting of the underlying genetic basis of the disease treated in this trial. Further, mTOR inhibitors can act to control the dysregulation that leads to hyperkeratosis. As a result, the symptoms, such as pain and itch, can be reversibly ameliorated by therapeutic treatment as described herein.

It should be understood that the above-described methods are only illustrative of some embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that variations including, may be made without departing from the principles and concepts set forth herein.

What is claimed is:

1. A method of treating symptoms of pain and/or itch in a target region of a subject, comprising:
topically administering to the subject a therapeutically effective amount of an mTOR inhibitor.

2. The method of claim 1, wherein the targeted region is a local skin region.

3. The method of claim 2, wherein the local skin region includes a condition producing the pain and/or itch symptom.

4. The method of claim 3, wherein the condition is a member selected from the group consisting of: a keratosis, pachyonychia congenita, epidermolysis bullosa, hyperhidrosis, a wart, a callus, dermatitis, and psoriasis.

5. The method of claim 3, wherein the condition is an injury.

6. The method of claim 5, wherein the injury is a member selected from the group consisting of: burns, abrasions, bites, surgical procedures, and chemical reactions.

7. The method of claim 2, wherein the local skin region is located away from a condition producing the pain and/or itch symptom.

8. The method of claim 1, wherein the target region is located inside the subject.

9. The method of claim 8, wherein the topical administration is transdermal administration.

10. The method of claim 1, wherein the target region is systemic.

11. The method of claim 1, wherein the therapeutically effective amount is an amount sufficient to deliver from 1 mg/day to 100 mg/day to the subject.

12. The method of claim 1, wherein the therapeutically effective amount is from 0.05 wt % to 5 wt %.

13. The method of claim 1, wherein the mTOR inhibitor includes a member selected from the group consisting of: rapamycin, temsirolimus, everolimus, AP-23573, AP-23481, derivatives thereof, and combinations thereof.

14. The method of claim 1, wherein the mTOR inhibitor is rapamycin or an analogue thereof.

15. A method of treating symptoms of pain and/or itch in a target region of a subject, comprising:
topically administering to the subject a topical dosage form comprising a therapeutically effective amount of an mTOR inhibitor in a delivery vehicle comprising:
about 0.05 wt % to about 5 wt % rapamycin;
about 0.05 wt % to about 1.0 wt % of a polymer having surfactant properties;
about 0.1 wt % to about 2 wt % of a polymer having thickening properties;
about 0.5 wt % to about 5 wt % of a glycol about 0.1 wt % to about 10 wt % of benzyl alcohol;
about 0.2 wt % to about 3 wt % of a C₁₀-C₂₀ fatty acid;
about 55 wt % to about 98 wt % water; and
about 0.01 wt % to about 0.5 wt % of a base.

16. A topical formulation for treating symptoms of pain and/or itch in a target area of a subject manifesting such symptoms comprising:
a therapeutically effective amount of an mTOR inhibitor;
an amount of a polymer having surfactant properties;
an amount of a polymer having thickening properties;
an amount of a solvent for solubilizing the mTOR inhibitor;
an amount of glycol;
an amount of C₁₀-C₂₀ fatty acid;
an amount of a base; and
an amount of water.

17. The topical formulation of claim 16, wherein the polymer having surfactant properties is an acrylate C₁₀-C₃₀ alkyl acrylate crosspolymer.

18. The topical formulation of claim 16, wherein the polymer having thickening properties is a hydrophobically modified cross-linked acrylate copolymer.

19. The topical formulation of claim 16, wherein the solvent for solubilizing the mTOR inhibitor is benzyl alcohol.

20. The topical formulation of claim 16, wherein the glycol is a member of the group consisting of butylene glycol, propylene glycol, diethylene glycol, triethylene glycol, ethylene glycol monomethyl ether, or other glycols and glycol ethers, and combinations thereof.

21. The topical formulation of claim 16, wherein the C₁₀-C₂₀ fatty acid is a member of the group consisting of oleic acid, arachidonic acid, linoleic acid, linolenic acid, or other fatty acids or combinations thereof.

22. The topical formulation of claim 1, wherein the base is a member of the group consisting of triethanolamine, tetrasodium ethylenediaminetetraacetic acid, alkali metal hydroxides such as sodium hydroxide and combinations thereof.

23. The topical formulation of claim 1, further comprising an emollient.

24. The topical formulation of claim 23, wherein the emollient is a member of the group consisting of mineral oil, dimethicone, and combinations thereof.

25. The topical formulation of claim 18, further comprising about 0.1 wt % to 5 wt % of an emollient.

26. The topical formulation of claim 25, wherein the emollient is mineral oil.

27. A pain and/or itch treating topical dosage form comprising;
an amount of an mTOR inhibitor that is sufficient to treat pain and/or itch in a target region of a subject in a vehicle suitable for topical administration to the subject.
